(19) United States
(12) Patent Application Publication
(10) Pub. No.: US 2010/0217123 A1
(43) Pub. Date: Aug. 26, 2010

(54) METHODS AND SYSTEMS OF MANAGING ULTRASONOGRAPHIC DIAGNOSIS

(76) Inventors: Aharon Eran, Herzlia (IL); Elad Eran, Herzlia (IL)

Correspondence Address: MARTIN D. MOYNIHAN d/b/a PRTSI, INC., P.O. BOX 16446, ARLINGTON, VA, 22215, US

(21) Appl. No.: 12/710,393

(22) Filed: Feb. 23, 2010

Related U.S. Application Data
(60) Provisional application No. 61/202,357, filed on Feb. 23, 2009.

(51) Int. Cl. A61B 8/00

(52) U.S. Cl. 600/437

(57) ABSTRACT

A method of monitoring ultrasonographic fetal organ screening. The method comprises receiving a plurality of ultrasonographic images captured by an ultrasonographic probe during an organ screening of a fetus, automatically identifying a group of ultrasonographic images, where each member of the group depicts one of a plurality of fetal scan planes, presenting the members of the group to an operator and receiving a diagnosis of each member in response, and monitoring the diagnosis to verify that each fetal scan plane is diagnosed.

### RELATED APPLICATION/S

This application claims priority from U.S. Patent Application No. 61/202,357, filed on Feb. 23, 2009. The content of the above document is incorporated by reference as if fully set forth herein.

### FIELD AND BACKGROUND OF THE INVENTION

The present invention, in some embodiments thereof, relates to medical diagnosis management and, more particularly, but not exclusively, to system and method of managing ultrasonographic diagnosis of fetal anatomy and measure.

The Ultrasound technology changed our way of life. Now in its third decade, organ screening by ultrasound is an accepted and common test method of pregnant women. Since this system of testing became widely used, it changed the world of gynecology. There are few decisions in the period of pregnancy that are not based upon organ screening. The organ screening goals are to confirm the pregnancy age and the healthy development of the fetus and to detect fetal abnormalities. One of the major advantages of the organ screening is an early detection of the abnormalities which enable the obstetrician to decide on the termination of pregnancy, or curing the fatality.

In the organ screening, we are able to detect abnormalities in the brain, head, heart, spine, urinary system, liver, limbs, digestion system and in effect in every part of the fetus body. This screening is done by an expert obstetrician that is trained to this check specifically, and is supposed to detect the abnormalities in the fetus.

Various methods and systems have been developed to assist in the ultrasonographic screening process. For example U.S. Pat. No. 7,343,190 filed on Jul. 28, 2004 describe method and system for assessing fetal abnormality based on landmarks. According to one embodiment, at least two coordinates are received for each of a plurality of points identifying a configuration of landmarks in a fetal image, and any of the received coordinates of any of the plurality of points are utilized as markers to assess fetal abnormality. According to another embodiment, at least two coordinates are received for each of a plurality of points identifying a configuration of landmarks in a fetal image, and one or more values resulting from a linear combination of any of the received coordinates of any of the plurality of points are utilized as markers to assess fetal abnormality.

### SUMMARY OF THE INVENTION

According to an aspect of some embodiments of the present invention there is provided a method of monitoring ultrasonographic fetal organ screening. The method comprises receiving a plurality of ultrasonographic images captured by an ultrasonographic probe during an organ screening of a fetus, automatically identifying a group of the plurality of ultrasonographic images, each member of the group depicting a fetal scan plane selected from a plurality of fetal scan planes, presenting the group to an operator and receiving a diagnosis of each the member in response, and monitoring the diagnosis to verify each the fetal scan plane being diagnosed.

Optionally, the identifying comprises identifying in each the member at least one fetal organ segment and classifying each the member as a certain of the plurality of fetal scan planes according to an analysis of the at least one fetal organ segment.

Optionally, the identifying comprises registering each the ultrasonographic image according to at least one reference map depicting at least one of the plurality of fetal scan planes.

Optionally, the identifying comprises evaluating at least one difference between the at least one fetal organ segment and at least respective fetal organ segment in at least one reference map depicting at least one of the plurality of fetal scan planes.

Optionally, the method further comprises aligning the plurality of ultrasonographic images before the identifying.

Optionally, the receiving comprises receiving the plurality of ultrasonographic images while the ultrasonographic probe being manually maneuvered by a human operator over the uterus of a patient.

Optionally, the plurality of fetal scan planes are selected from of a group consisting of: a sagittal cerebral plane, a coronal cerebral plane, an axial transventricular plane, an axial transcerebellar plane, a four chamber cardiac view, a cardiac outflow tract, a great artery abdomen transverse cardiac plane, an transverse abdomen plane, a fetal spine plane, and a plane passing through the longitudinal axis of a fetal limb.

Optionally, the identifying comprises identifying the member as an image depicting a cerebral coronal plane by identifying a plurality of segments depicting the brain cavum septum pellucidum (CSP), the corpus callosum, and he anterior horns of the third ventricle of the brain of the fetus therein.

Optionally, the identifying comprises identifying the member as an image depicting a cerebral coronal plane by segmenting the brain cavum septum pellucidum (CSP) and the cavum of the brain of the fetus therein.

Optionally, the identifying comprises identifying the member as an image depicting a cerebral axial plane by segmenting the brain cavum septum pellucidum (CSP) and the Thalamus of the brain of the fetus therein.

Optionally, the identifying comprises identifying the member as an image depicting a cerebral axial plane by segmenting the Cerebellum and the Cisterna Magana of the brain of the fetus therein.

Optionally, the identifying comprises identifying the member as an image depicting a spinal axis plane by segmenting a plurality of vertebral bodies therein.

Optionally, the identifying comprises identifying the member as an image depicting a four-chambers plane by segmenting four heart chambers therein.

According to an aspect of some embodiments of the present invention there is provided a method of managing ultrasonographic fetus organ screening. The method comprises receiving a plurality of ultrasonographic images captured by an ultrasonographic probe performing an organ screening of a fetus, automatically identifying a group of the plurality of ultrasonographic images, each member of the group depicting a fetal scan plane selected from a plurality of fetal scan planes, and for each the member generating and presenting a form so as to allow an operator to submit a diagnosis of a respective the member.

According to an aspect of some embodiments of the present invention there is provided a system of managing ultrasonographic fetus organ screening. The system comprises an interface which receives a plurality of ultrasonographic images captured by an ultrasonographic probe performing an organ screening of a fetus of a patient, an plane selection module which automatically identifies a group of the plurality of ultrasonographic images, each member of the group depicting a fetal scan plane selected from a plurality of fetal scan planes, and a form generator which generates a plurality of forms each for a respective the member, each the form being adapted to a fetal scan plane depicted by the respective member.

Optionally, the system comprises a presentation unit which generates an alert if the group does not include ultrasonographic images depicting all the plurality of fetal scan planes.

Optionally, the system comprises a user interface for allowing an operator to fill in the plurality of forms.

Optionally, the form generator monitors the filling in and outputs an indication about the completeness of the filling in.

Optionally, the system comprises a database having a plurality of reference maps of a plurality of normal fetal scan planes, the plane selection module compares between the plurality of reference maps and the plurality of ultrasonographic images to identify the group.

More optionally, each the reference map is associated with a different group of at least one of patients, organ screenings, and fetuses, the plane selection module compares the plurality of ultrasonographic images according to at least one of the organ screening, the fetus and the patient.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a system of managing ultrasonographic fetal organ screening procedure, according to some embodiments of the present invention;

FIG. 2A is a flowchart of a method of managing ultrasonographic fetal organ screening, for example using the system of FIG. 1, according to some embodiments of the present invention;

FIG. 2B is a flowchart of a method of segmenting and matching an ultrasonographic image according to some embodiments of the preset invention;

FIG. 3 is an exemplary form which is presented to allow the operator, to input data, according to some embodiments of the present invention;

FIG. 4 is a schematic illustration of sub modules of the analysis module of the system of FIG. 1, according to some embodiments of the present invention;

FIG. 5 is a flowchart of a method of identifying and classifying an ultrasonic image as an image depicting the coronal plane of the brain of a fetus, according to some embodiments of the present invention;

FIGS. 6A and 6B are images of a cerebral Coronal plane wherein the longitudinal fissure, a longitudinal line, separation line between the left and the right hemispheres of the brain, the CSP, and the left and right ventricular horns are segmented;

FIG. 7 is a flowchart of a method of identifying and classifying an ultrasonic image as an image depicting the coronal plane of the brain a fetus, according to some embodiments of the present invention;

FIG. 8, which is a flowchart of a method of identifying and classifying an image as depicting in the cerebral axial plane of a fetus, according to some embodiments of the present invention;

FIG. 9 is an image depicting the transthalamic line, the CSP and the brain Thalamus segments in an image of a cerebral plane;

FIG. 10 is a flowchart of a method of identifying and classifying an ultrasonic image as an image depicting the cerebral axial plane of a fetus, according to some embodiments of the present invention;

FIG. 11 is a flowchart of a of identifying and classifying an ultrasonic image as an image depicting cerebral axial plane of a fetus, according to some embodiments of the present invention;

FIG. 12 is an image depicting the Cerebellum and the Cisterna Magana segments in an image of a cerebral plane;

FIG. 13 is a flowchart of a of identifying and classifying an ultrasonic image as an image depicting cerebral axial plane of a fetus, according to some embodiments of the present invention;

FIG. 14 is a flowchart of another method of identifying and classifying an ultrasonic image as an image depicting a cerebral axial plane of a fetus, according to some embodiments of the present invention;

FIG. 15 is a flowchart of a process of identifying and classifying an ultrasonic image as an image depicting a cardiac plane, according to some embodiments of the present invention;

FIGS. 16 and 17 are images depicting a four-chamber plane of a fetal heart and respective segments thereof;

FIG. 18 is a flowchart of a process of identifying and classifying an ultrasonic image as an image depicting an abdominal plane, according to some embodiments of the present invention;

FIG. 19 is an image depicting an abdominal plane of a fetus and respective segments thereof;

FIG. 20, which is a flowchart of a process of identifying and classifying an ultrasonic image as an image depicting a plane passing through a limb, according to some embodiments of the present invention; and

FIGS. 21-23 are images depicting a limb plane of a fetus and the longitudinal axis of the respective limb.

### DESCRIPTION OF EMBODIMENTS OF THE INVENTION

The present invention, in some embodiments thereof, relates to medical diagnosis management and, more particularly, but not exclusively, to system and method of managing ultrasonographic diagnosis of fetal anatomy and measure.

According to some embodiments of the present invention there is provided system and method of monitoring ultrasonographic fetal organ screening and/or the diagnosis of the ultrasonographic images. The method is based on ultrasonographic images which are captured by an ultrasonographic probe during an organ screening of a fetus, for example at the 21-23 week of pregnancy. This allows selecting a group of ultrasonographic images which depict selected fetal scan planes, such as the fetal scan planes which are diagnosed during a routine organ screening procedures, for example the cerebral Sagittal plane, the cerebral Coronal plane, the axial transventricular plane, the axial transcerebellar plane, the cardiac four chamber view, the cardiac outflow tract, the cardiac great artery abdomen transverse plane, the abdomen transverse plane (fetal kidneys), the fetal spine plane, and fetal limb plane(s). The group is selected automatically and/or semi automatically, optionally sequentially, for example as described below. Now, forms are generated, each for allowing an operator, such as a technician or a physician, to diagnose one of the planes according to a visual analysis of one of the selected images. The operator, for example, may estimate a presence or an absence of a fetal abnormality, such as a structural malformation, in the respective scan plane. Optionally, the identification and classification of images depicting selected fetal planes is performed by matching the received ultrasonographic images with reference maps, such as reference images which depict fetal scan planes of normal and/or abnormal fetuses. Optionally, the reference map is generated by a statistical analysis of a plurality of studies, of fetal scan planes of a plurality of fetuses.

The system utilizes the similarity of the anatomy among fetuses at the same age of development for detecting fetal scan planes of ultrasonographic images. The matching between anatomic characteristics of an ultrasonographic image and the anatomic characteristics of reference map allows determining whether the ultrasonographic image depicts the fetal scan plane documented in the reference map or not.

This process allows monitoring the diagnosis process and to verify that each one of the fetal scan planes is fully analyzed and/or diagnosed.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

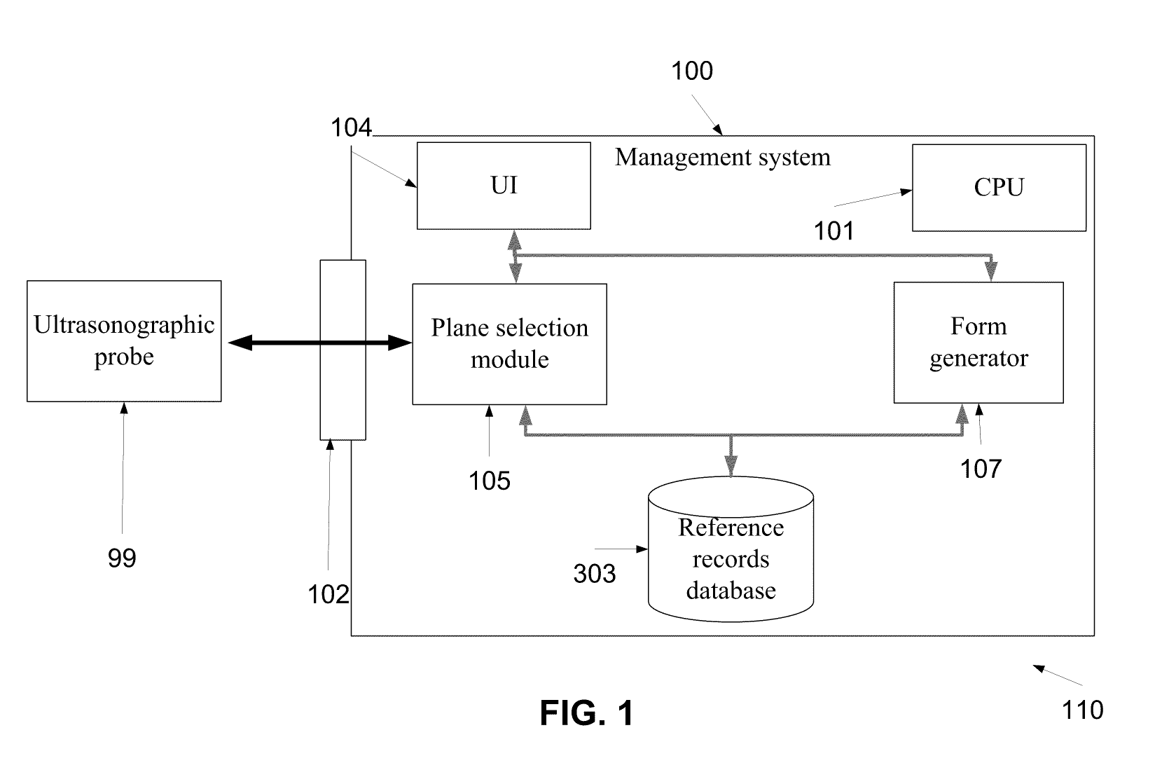

Reference is now made to FIG. 1, which is a system 100 of managing ultrasonographic fetal organ screening procedure, according to some embodiments of the present invention. The system 100 may be implemented using a computing unit that includes a processor 101, an input device 102, a presentation unit 103, such as a screen, and a memory. The input device 102 includes an interface set to receive a plurality of ultrasonographic images from an ultrasonographic probe 99 that is used to perform an organ screening of a fetus. The ultrasonographic probe 99 may be a two-dimensional layout ultrasonographic probe, an arc layout ultrasonographic probe and/or any other probe which may be used for capturing fetal images.

Optionally, the system 100 further comprises a user interface (UI) 104 that receives instructions from a user, for example via a man machine interface (MMI) device, such as a keyboard, a mouse, a touch screen, and the like, and a presentation unit for displaying, among others, the images of the plurality of ultrasonographic images.

The system 100 further includes a plane selection module 105 which identifies a group of ultrasonographic images which depict selected fetal scan planes from the received ultrasonographic images. Optionally, the plane selection module 105 identifies images depicting a set of fetal scan planes automatically by comparing the received ultrasonographic images with reference maps, such as images, stored in a database 303, to identify a match. When a match is found, the matching ultrasonographic image is classified as depicting a fetal scan plane also depicted in the matching image of one of the reference records. The database 303 hosts a collection of reference records or maps, such as reference images, referred to herein as reference records. Each reference record maps fetal organs in a certain fetal scan plane. For example, a reference record may include a map of fetal segments and/or one or more exemplary segmented images with fetal organ segments. In another example, the reference record includes a list of one or more fetal organ segments each with a coordinates of a relative location of the respective organ in the fetal scan plane.

Optionally, the reference record is generated according to data collected from a plurality of ultrasonographic images of fetuses having similar characteristics, such as age. The combination of the plurality of ultrasonographic images allows creating a statistical model that reflects common parameters of a fetal scan plane of healthy fetuses. The combination may of 2, 4, 8, 16, 32, 64, 128, 256, 1000, 10,000 or any intermediate or higher number of ultrasonographic images of fetuses having similar characteristics.

Optionally, different reference records are statistically generated according to images of patients having common medical data, such as age. In such a manner, the reference record used for segmentation and/or classification is based on images of patients that share medical data with the imaged patient.

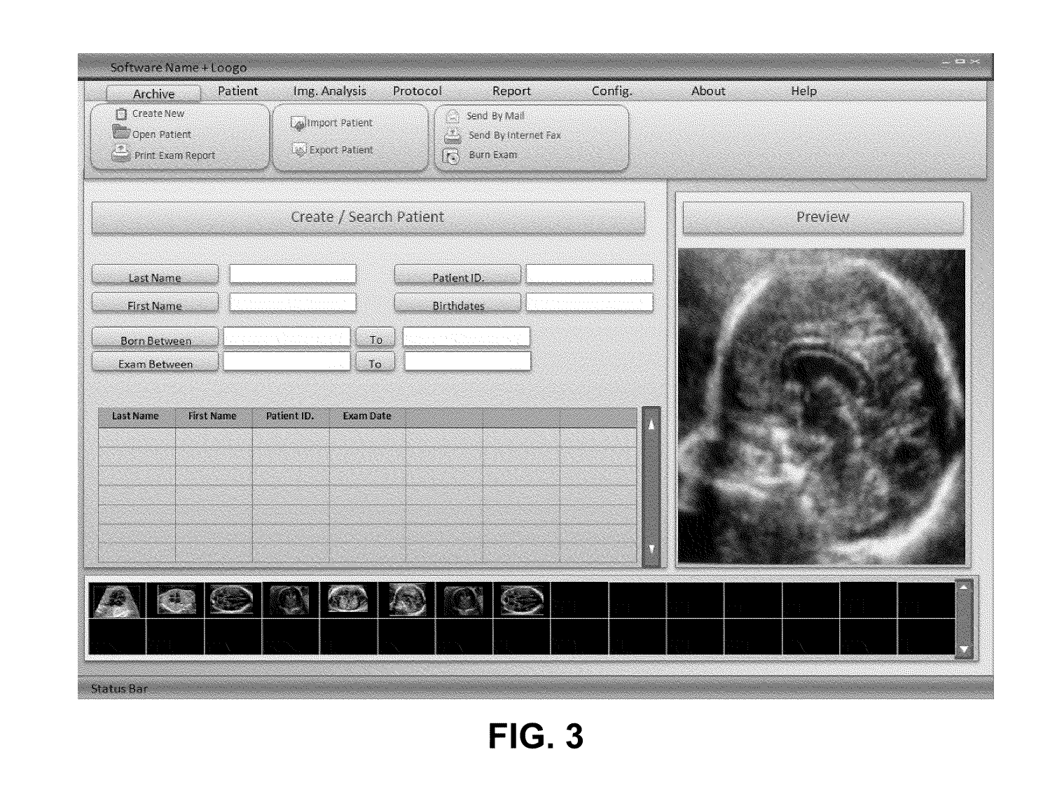

Optionally, the system 100 includes a form generator 107 which generates a form for each fetal scan plane in the group. Each form allows the operator to submit his diagnosis pertaining to the fetal scan plane. Optionally the form is a graphical user interface which is presented to the operator in a client terminal, such a personal computer, a PDA, a Smartphone, a thin client, a laptop and the like. FIG. 3 is an exemplary form which is presented to allow the operator, to input data pertaining to the presence and/or absence of one or more fetal abnormalities, such as structural malformations, in relation to an ultrasonographic image depicting a selected scan plane. The operator may mark the fetal abnormalities and the level of certainty thereof. The form generator 107 allows monitoring the inputs provided by the operator. Optionally, a set of fetal scan planes are defined per examination. Each fetal scan plane is associated with a checklist of one or more diagnosis events. The form generator 107 generates a form with rubrics according to the respective checklist, for example as shown at FIG. 3. Optionally, each form is generated automatically after an ultrasonographic image is identified as depicting a certain fetal scan plane during an ultrasonographic fetal examination. Optionally, the form generator 107 instructs the user which fetal scan plane should be diagnosed next, allowing maneuvering the ultrasonographic probe accordingly. In such a manner, the form generator 107 assures that all the fetal scan planes are diagnosed. Optionally, the form generator 107 generates an alarm if not all the fetal scan planes have been verified and/or if not all the rubrics have been filled in. Optionally, the operator may use the UI 104 to change or determine the order in which the fetal scan planes being diagnosed.

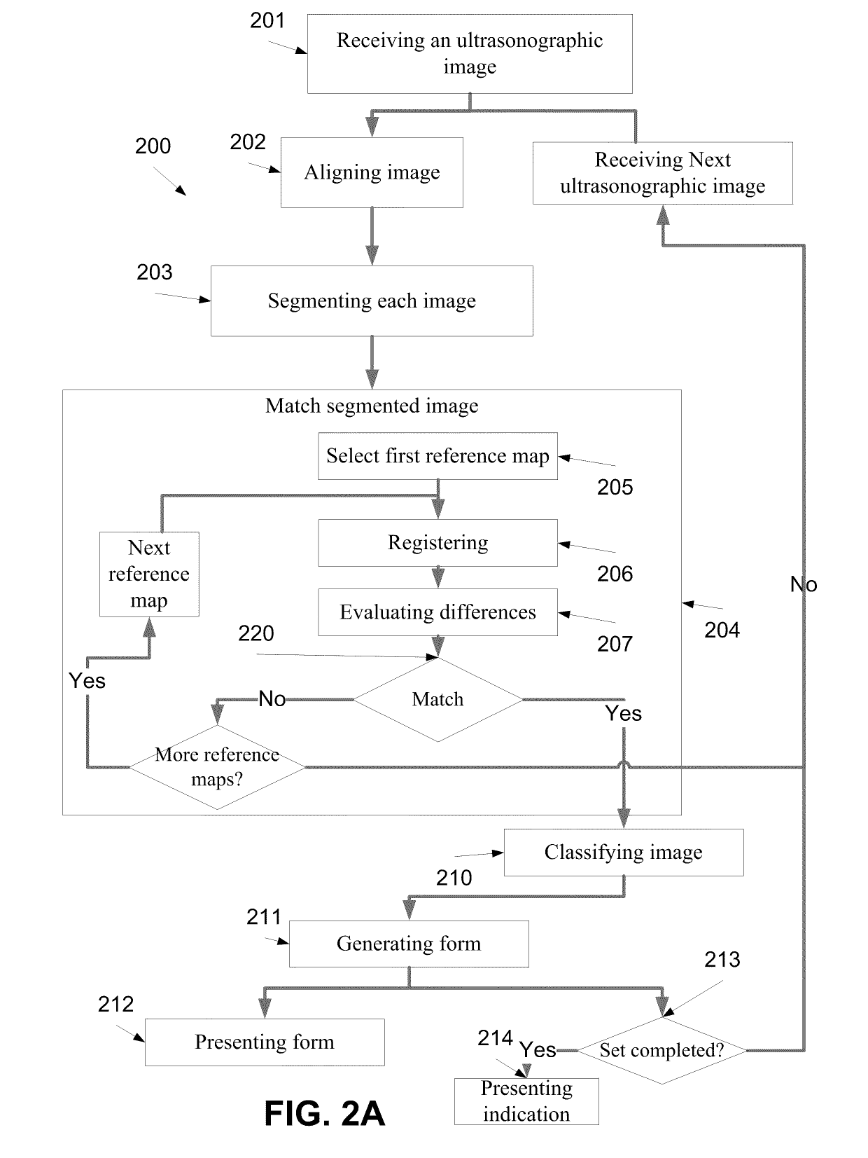

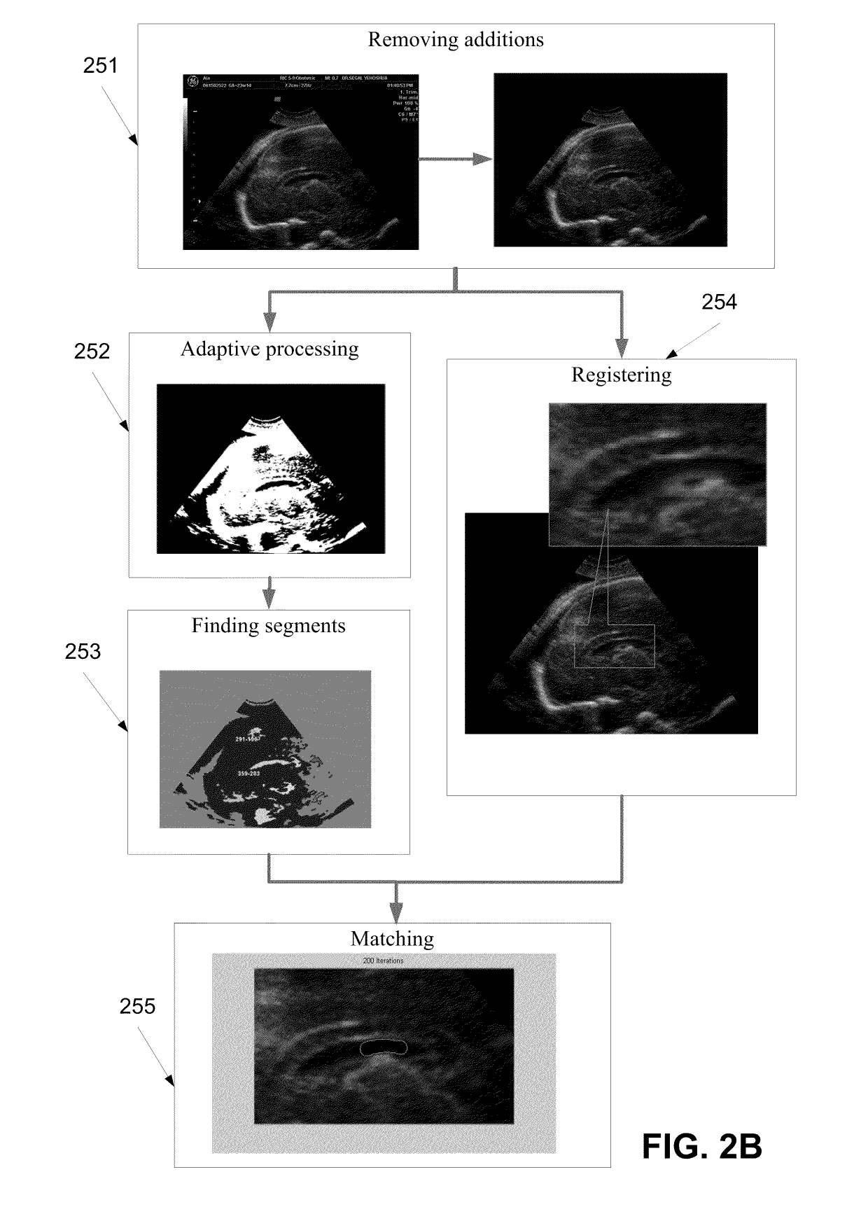

Reference is also now made to FIG. 2A, which is a flowchart 200 of a method of identifying an ultrasonographic image depicting a selected fetal scan plane from a plurality of fetal scan planes during an ultrasonographic fetal organ screening, for example using the system 100, according to some embodiments of the present invention. The plurality of fetal scan planes includes some or all of the following planes fetal scan planes: cerebral Sagittal plane, cerebral Coronal plane, axial transventricular plane, axial transcerebellar plane, cardiac four chamber view, cardiac outflow tract, cardiac great artery abdomen transverse plane, abdomen transverse plane (fetal kidneys), fetal spine plane, fetal hand plane(s), and a plane passing through the longitudinal axis of fetal leg(s).

Optionally, in use, an operator, such as a technician or a physician, manually maneuvers the ultrasonographic probe on the abdomen of a pregnant woman. Similarly to the commonly known in the art, screening procedures, the ultrasonographic images which are captured by the ultrasonographic probe, are presented to the operator, for example on the presentation unit of the UI 104. In another embodiment, the probe is automatically maneuvered by a robotic hand. As shown at 201, the plurality of ultrasonographic images which are captured by the ultrasonographic probe 99 during the organ screening of a fetus, are received by the input unit 102. Optionally, some of the ultrasonographic images are selected by the operator as potential images. In such an embodiment, these images are verified as candidates of images depicting the selected fetal scan planes.

Each received image 202, or candidate image, is aligned according to a set of rules used to align the reference maps.

Than, the received image 203 is segmented. During the process one or more fetal organ segments are identified and marked in the received image, for example as described below. Optionally, the segmentation is performed using a set of segmentation rules and/or according to a match with segmentation templates, for example as described below.

As shown at 204 and outlined above, the segmented image is matched with some or all of the reference maps in the database. The segmented image is matched with each reference map and/or each selected reference map. Each one of the reference maps depicts a typical representation of organs in a certain fetal scan plane in a certain age of a healthy fetus, such as a typical cerebral fetal sagittal plane of a healthy fetus in the 21 week of pregnancy. The map may be an exemplary image. Optionally, the matching is based on a number of actions. First, one of the reference records is selected, as shown at 205. Optionally, only a group of reference maps are matched with the image. Optionally, the reference maps are selected according to a match between the fetus characteristics, the carrying patient characteristics, and/or the screening type characteristics. For example as described above. Optionally, the plane selection module 106 selects a group of reference maps to match from the database 303 according to data, such as the age of the fetus, the type of the ultrasonographic fetal organ screening procedure, the age of the fetus, the type of the ultrasonographic probe and the like. The data may be inputted manually, for example according to inputs of the operator which are received manually, via the UI 104, or automatically, for example by extracting data associated with the image. By choosing a suitable reference record, certain causes of faulty classification may be avoided. Such a faulty classification occurs as the same pathology may have different indications and expressions in different age of the fetus.

For each matched map, the matching optionally includes registering between the received image 202 and each one of the matched reference maps using a 2D affine transformation, for example as depicted in 206. Optionally, an affine transformation vector is estimated according to the positioning differences between the images. Optionally, the registration is performed by an optic flow process for estimating an optic-flow field according to positioning differences between the segments of the received image and the segments defined in the matched reference map.

Now, as shown at 207, the differences between the segments are evaluated. If the differences are below a certain threshold, a match is found, else no match is found and another reference image is matched. This process is repeated iteratively until all the reference maps are matched.

For example, reference is now made to FIG. 2B, which is a flowchart of a method of segmenting and matching an ultrasonographic image according to some embodiments of the preset invention. Each block includes an exemplary image depicting an exemplary outcome of applying it on an exemplary ultrasonographic image. First, as shown at 251, marking, such as text and tags are removed. Then, as shown at 252, adaptive processes, such as noise filtering, adaptive thresholding, binarization and the like are performed. Than, as shown at 253, objects in the ultrasonographic image are identified and segmented. As shown at 254 the image is registered with the matched reference map. Now, the registered segments in the received image may be matched with the respective organ segments which are marked in the matched reference map, as shown at 255. Optionally, active contour process is used to refine the matching process.

Additionally or alternatively, the matching is performed by verifying the compliance of each segment to one or more rules or instructions. For example, the estimated shape, length, area, width-length ratio and/or any other characteristic of a healthy or pathological organ or sub organ may be specified to allow verifying the absence or presence of certain abnormality in relation to the fetal organ segment. In such an embodiment, the matching is performed with reference records and not reference maps wherein each reference record is associated with a list of rules.

If a match is found, as shown at 208, the matched image is classified according to the matching image. The classification indicates which fetal scan plane is depicted in the image. Optionally, some of the reference maps are of fetal scan planes depicting one or more abnormalities. When a match with such a reference map is identified, the image is classified as abnormal. If no match is found, as shown at 209, the next reference map or selected reference map is processed according to blocks 206-207. This process is iteratively repeated until a match is found or until all the reference maps have been matched.

Now, as shown at 211 a form is generated and presented to the operator, as shown at 212. The form allows the operator to diagnose the presence or absence of one or more fetal abnormalities in the respective fetal scan plane. Optionally, the form is generated according to specific characteristics of the scan plane depicted in the matched image, for example, potential abnormalities to diagnose and the like. The forms may be generated sequentially, each time after a matching image is found and/or simultaneously after images of all the fetal scan planes have been captured.

As shown at 213, this process is repeated iteratively until a set of images that depicts a set of selected fetal scan planes is matched.

Additionally or alternatively, as shown at 214, an indication is presented when images of all the fetal scan planes, which are required to be diagnosed during the ultrasonographic fetal organ screening procedure, and/or the analysis thereof have been captured. This allows constantly notifying the operator about the incompleteness and/or the completeness of the organ screening procedure and/or alerting operator if she tries to finish the organ screening procedure without all the scan planes.

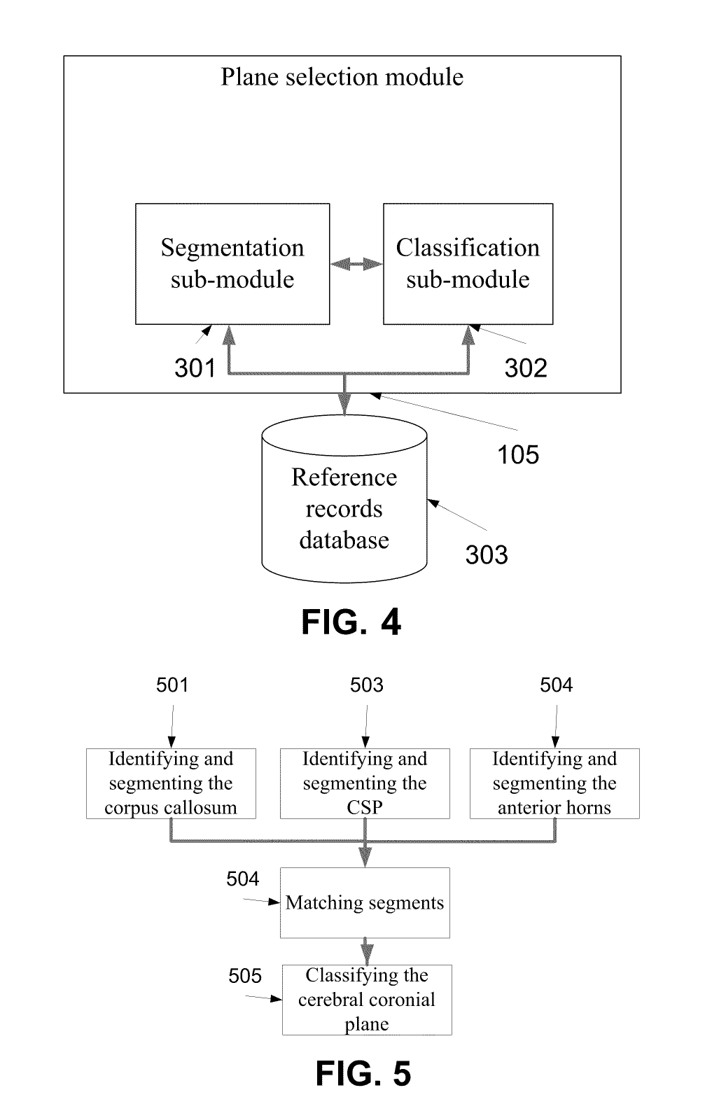

Reference is now made to FIG. 4, which is a schematic illustration of sub modules of the plane selection module 105, according to some embodiments of the present invention. The plane selection module 105 may include a segmentation sub-module 301 which receives the selected ultrasonographic images and identify one or more fetal organ segments therein, optionally according to segmentation instructions designated to the fetal scan planes having certain characteristics, such as fetal scan planes of a certain procedure and/or for a certain diagnosis. Such segmentation instructions are optionally taken from the database 303. Optionally, a fetal organ segment means a fetal organ, a sub organ having identifiable characteristics, a plurality of fetal body organs or sub organs, a fetus body system, a section of a fetus body system, an area in the fetus and/or a section of the fetal organ. The plane selection module 105 further comprises a classification sub-module 302 that receives the selected ultrasonographic image, optionally segmented, and/or with an indication of the depicted fetal scan plane and/or fetal organ segments, and classifies it, for example according to a matching process as described above. Optionally, the classification sub-module 302 outputs a classification of the depicted plane.

Reference is now made to a number of examples in which the plane selection module 105 classifies ultrasonographic images of different fetal scan planes, according to some embodiments of the present invention. The examples, depicted and described in FIGS. 5, 7, 8, 10, 11, 13-15, 18, and 20 may be performed using respective reference maps or records. For example, when an ultrasonographic image is matched with a reference map depicting abdominal plane, the segmentation may be performed according to characteristics of abdominal features. The methods may be sequentially performed on each received ultrasonographic image to facilitate the classification thereof. Optionally, one or more methods are selected in advance according to characteristics of the ultrasonographic image and/or of segments thereof. Failure to classify an image as a certain scan plane will induce the execution of another classification process. If all the classification processes have been failed, another image is probed, as shown in numeral 220 of FIG. 2A.

For example reference is now made to FIG. 5, which is a flowchart of a method of identifying and classifying an ultrasonic image as an image depicting the coronal plane of the brain of a fetus in the 21-23 week of pregnancy, according to some embodiments of the present invention.

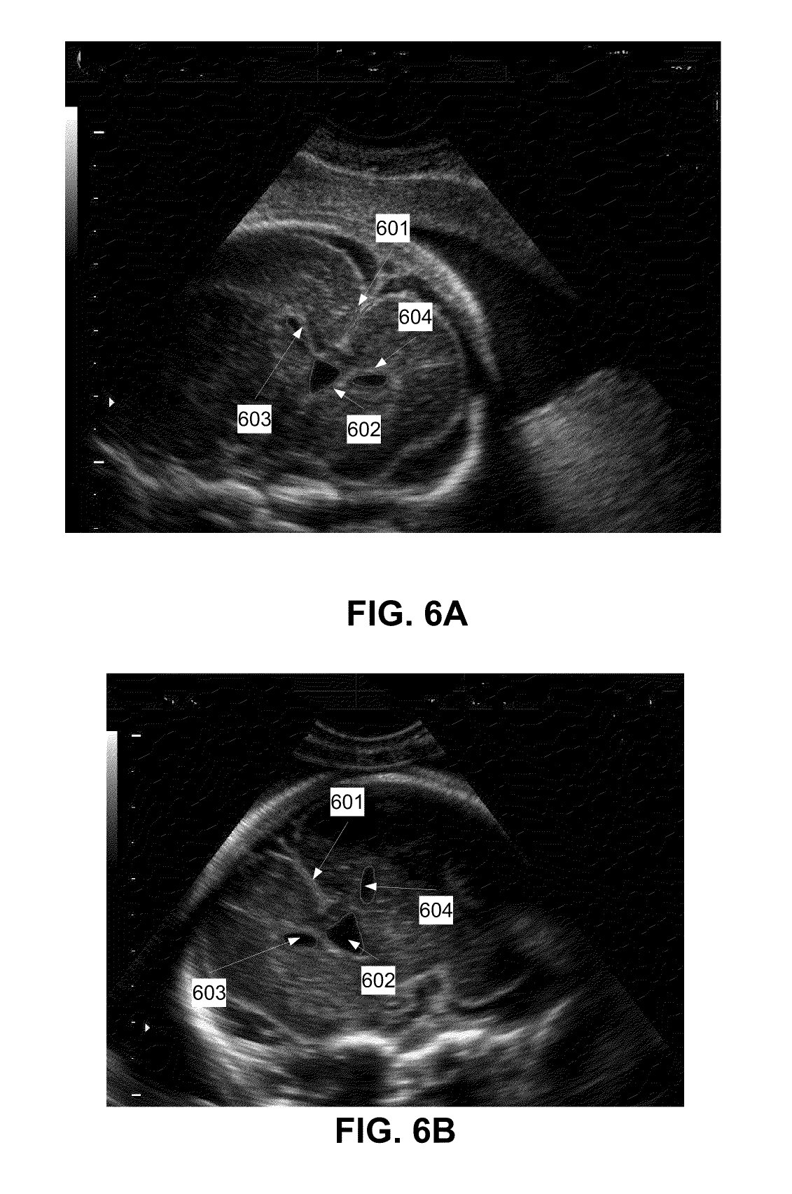

As shown at 501, the corpus callosum is identified and optionally segmented. As shown at 502, the brain cavum septum pellucidum (CSP) is identified and optionally segmented. As shown at 503, the anterior horns of the third ventricle of the brain is identified and optionally segmented. The identification and segmentation is performed by the segmentation sub-module 301, optionally according to rules defined in a respective reference record which is defined for the cerebral coronal plane. For example, FIGS. 6A and 6B are images of a cerebral coronal plane wherein the longitudinal fissure 601, a longitudinal line, separation line between the left and the right hemispheres of the brain, the CSP 602, and the left and right ventricular horns 603, 604 are segmented. Now, as shown at 504, these segments are matched with respective fetal organ segments in the matched reference map. For example, the corpus callosum segment may be matched with a map that depicts a healthy corpus callosum having a normal T shape. This match allows classifying an image as an image depicting the cerebral coronal plane of the fetus, as shown at 505.

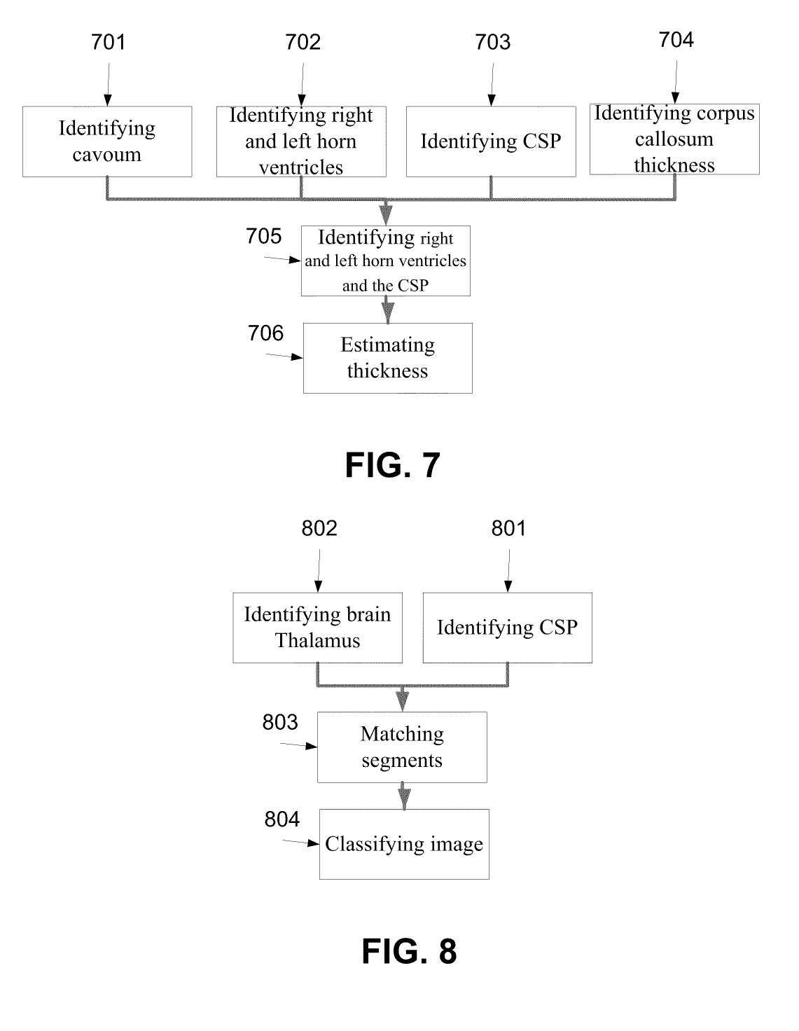

Additionally or alternatively, the compliance of each segment with one or more rules is verified. For example, the shape, the length, the area, the width-length ratio and/or any other characteristic may be estimated and checked according to one or more instructions defined in the selected reference record. For example, a reference is now also made to FIG. 7, which is a flowchart of a method of classifying an ultrasonic image as an image depicting the coronal plane of the brain of a fetus in the 21-23 week of pregnancy, according to some embodiments of the present invention using one or more estimation rules.

First, as shown at 701, the location of the cavum is identified in the received image, for example using image processing and/or statistical learning algorithms. Than, as shown at 702, the right and left horn ventricles of the brain are identified, for example according to the shape of the respective segments. Than, as shown at 703, the CSP is detected. Now, as shown at 704, the thickness of the corpus callosum is estimated, for example by measuring the distance between the left and right contour lines of the respective segment. Now, as shown at 705 and 706, if the cavum, the right and left horn ventricles, and the CSP are identified and the thickness is estimated a predefined range, for example as required according to the respective medical standards, the image is classified as an image depicting a cerebral coronal plane.

Reference is now made to FIG. 8, which is a flowchart of a method of identifying and classifying an ultrasonic image as depicting a cerebral axial plane of a fetus in the 21-23 week of pregnancy, according to some embodiments of the present invention.

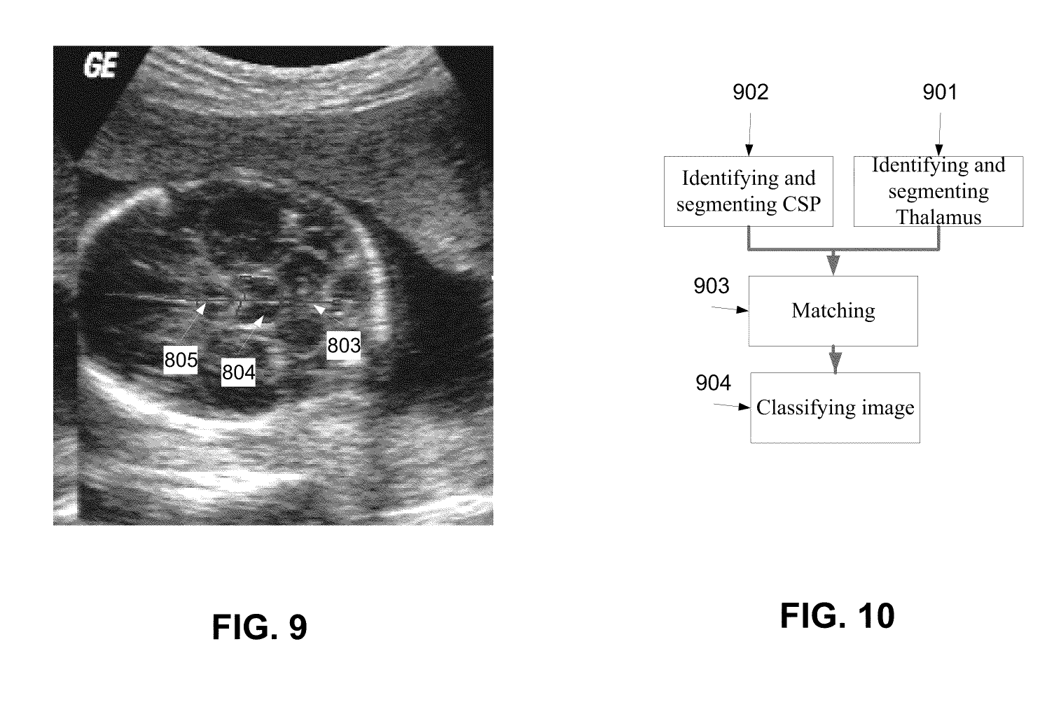

As shown at 801, the CSP is identified and optionally segmented. As shown at 802, the brain Thalamus is identified and optionally segmented. Than, as shown at 803, these segments are matched with respective segments in a selected reference record, for example, similarly to the described above. FIG. 9 depicts the transthalamic line 803 and the CSP and the brain Thalamus segments 804, 805.

Additionally or alternatively, the compliance of each segment with one or more rules is verified. For example reference is now made to FIG. 10, which is a flowchart of a method of classifying an ultrasonic image as an image depicting the cerebral axial plane of a fetus in the 21-23 week of pregnancy, according to some embodiments of the present invention. First, as shown at 901, 902 the CSP and the thalamus are identified and segmented in the matched image found using image processing and/or statistical learning algorithms. Now, as shown at 903, a match to respective segments is determined according to the outputs of the image processing and/or statistical learning algorithms and/or as described above. These algorithms are based on the ellipsoid shape of the Thalamus and round shape of the CSP. Accordingly, as shown at 904, the image is either classified as an image depicting the cerebral axial plane or not.

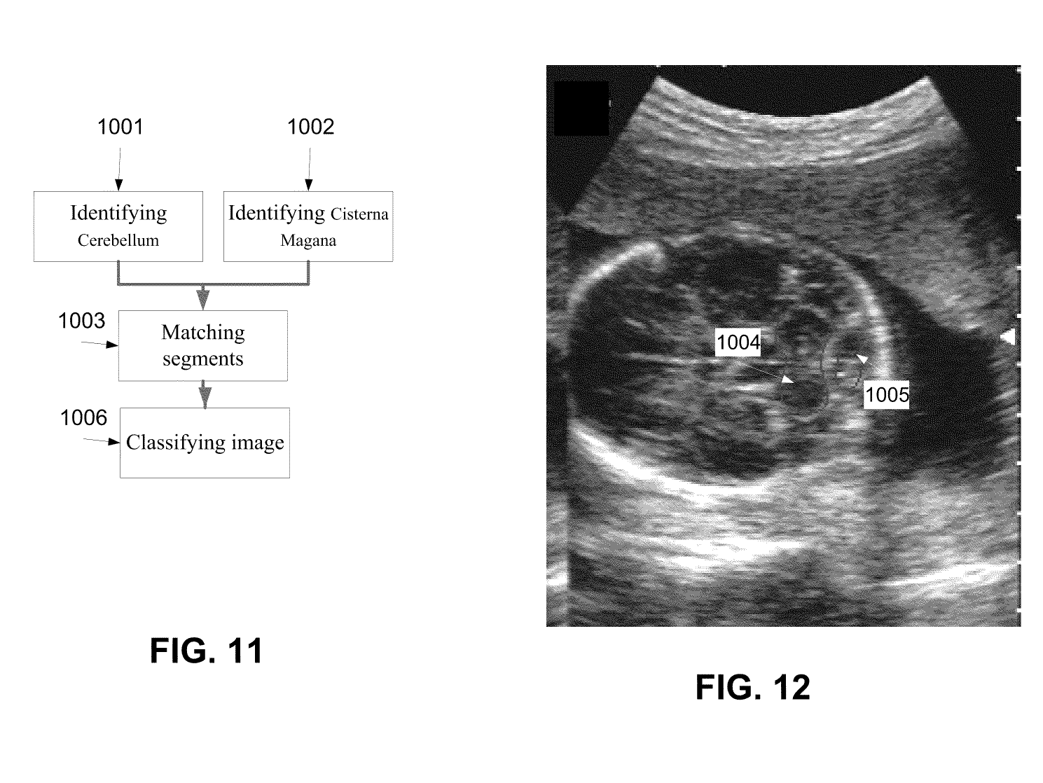

Reference is now made to FIG. 11, which is a flowchart of a method of identifying and classifying an ultrasonic image as an image depicting a cerebral axial plane of a fetus in the 21-23 week of pregnancy, according to some embodiments of the present invention.

As shown at 1001, the Cerebellum is identified and optionally segmented. As shown at 1002, the Cisterna Magana is identified and optionally segmented. Than, as shown at 1003, these segments are matched with respective segments in a selected reference record, for example, similarly to the described above. FIG. 12 depicts the Cerebellum and the Cisterna Magana segments 1004, 1005. This allows identifying and classifying an image as an image depicting the cerebral axial plane, for example as described above.

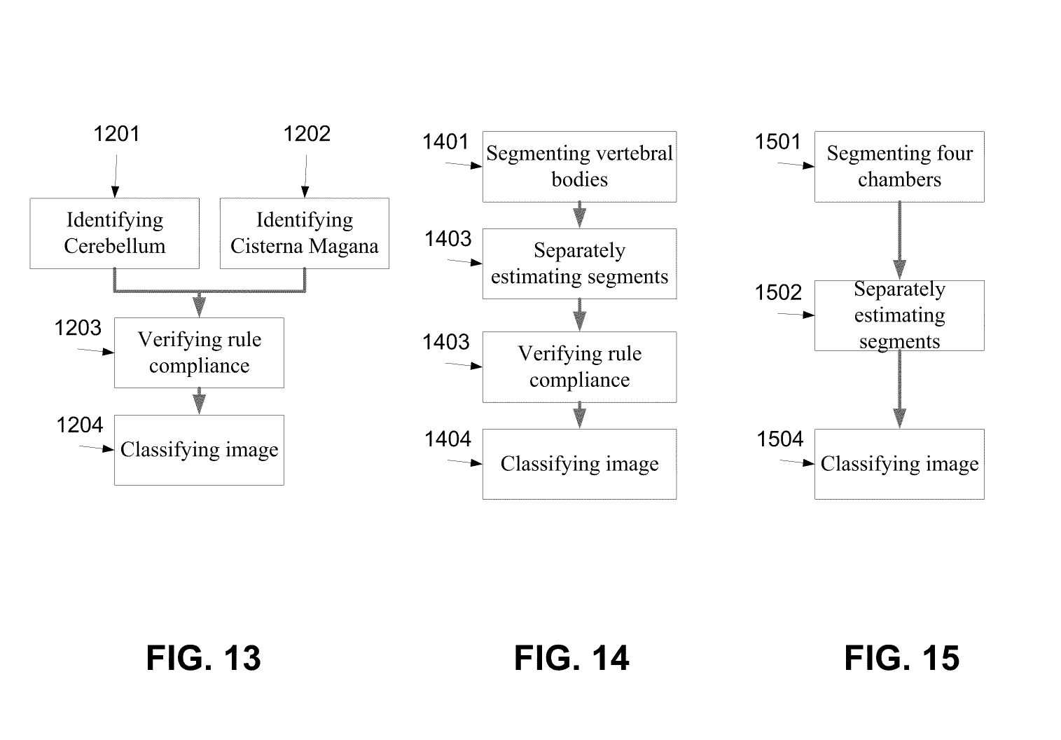

Additionally or alternatively, the compliance of each segment with one or more rules is verified. For example, reference is now made to FIG. 13, which is a flowchart of a method of identifying and classifying an ultrasonic image as an image depicting a cerebral axial plane of a fetus in the 21-23 week of pregnancy, according to some embodiments of the present invention. First, as shown at 1201, 1202, the segments depicting Cerebellum and Cisterna Magana are found in the matched image using image processing and/or statistical learning algorithms. As shown at 1203, the compliance of the Cerebellum and Cisterna Magana segments is checked. This allows verifying that the image depicts the cerebral axial plane according to the outputs of the image processing and/or statistical learning algorithms which are based on the shape and/or area of the segments. These algorithms are set to determine whether the segments have the ellipsoid shapes of the Cerebellum and Cisterna Magana segments. This allows classifying the image as an image depicting a cerebral axial plane, for example as described above and shown in 1204.

Reference is now made to an identification and classification of fetal planes which depict the spine. The spine may be evaluated in longitudinal, coronal and axial scan planes. For example, the analysis is based on a matched image that depicts a longitudinal scan plane that provides a longitudinal view passing through the spine, from the posterior portion to the anterior portion. Additionally or alternatively, an image that depicts a coronal plane that passes through the laminae is also a valid scan plane. Optionally, vertebral bodies are segmented and matched with respective segments in a reference map to identify a match with the respective scan plane. In another embodiment, the reference record defines the presence of 33 vertebral bodies. During the test these vertebral bodies are identified and optionally tested.

Reference is now made to FIG. 14, which is a flowchart of a method of identifying and classifying an image depicting a spinal plane of a fetus in the 21-23 week of pregnancy, according to some embodiments of the present invention. First, as shown at 1401, a plurality of vertebral bodies in a certain arrangement are found, and optionally counted, in an ultrasonographic image. Than, as shown at 1402, the vertebral bodies are separately measured or otherwise estimated. This allows, as shown at 1403, determining, for example according to characteristics of different vertebral bodies and/or their number, if the image depicts a suitable scan plane. This allows classifying an image as depicting the spinal cord of a fetus, as shown at 1404.

Reference is now made to FIG. 15, which is a flowchart of a process of determining whether an image depicts a cardiac plane, according to some embodiments of the present invention.

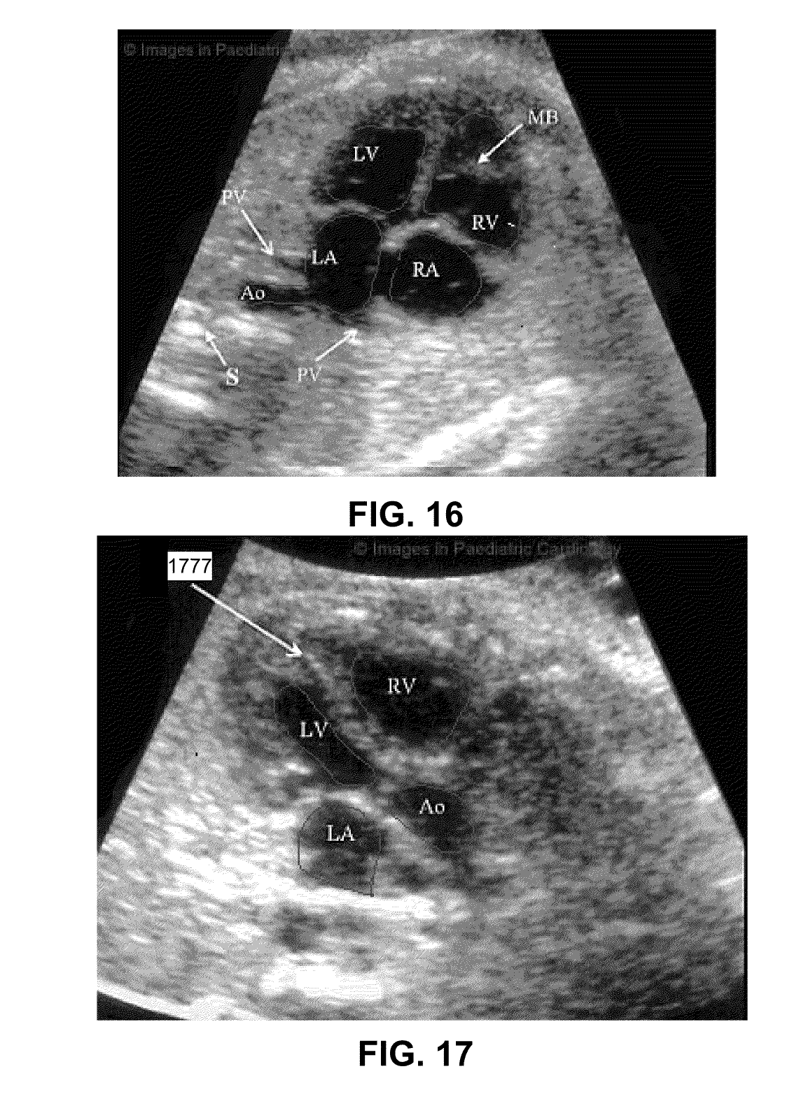

As shown at 1501, an ultrasonographic image is received and segmented. Optionally, four-chambers of the developing heart are segmented, left and right atriums and the left and right ventricles, for example as shown in FIG. 16. Now, as shown at 1502, each segment is separately matched with fetal organ segments in a matched respective record. As before, learning and image processing algorithms may be used for the match. This allows, as shown at 1504, classifying the image as an image depicting a fetal cardiac plane.

Additionally or alternatively, the presence or absence of a normal long axis aorta may be used to determine whether the image depicts the fetal cardiac plane or not. The determination may be set according to the arrangement of the segments and the distance between their contours, for example as shown in numeral 1777 of FIG. 17.

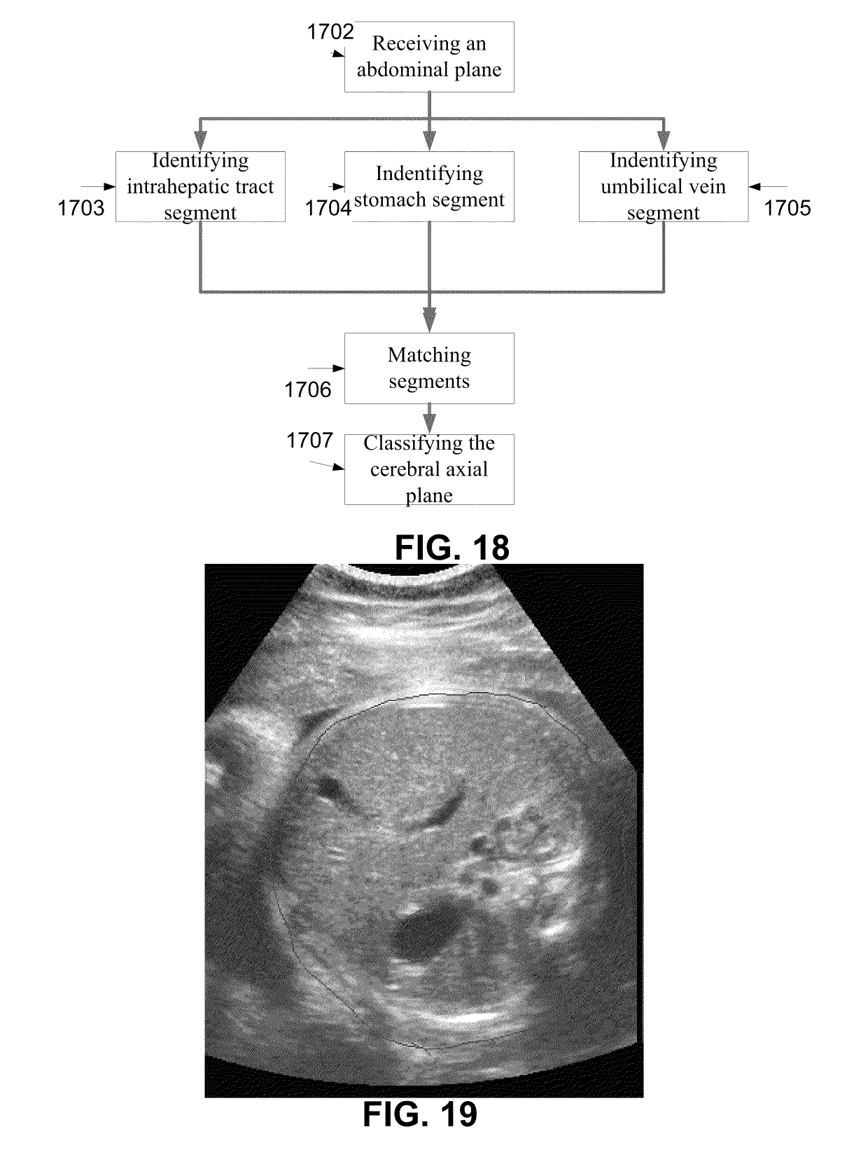

Reference is now made to FIG. 18, which is a flowchart of a process of identifying and classifying an image as an image depicting an abdominal plane, according to some embodiments of the present invention. First, as shown at 1702, the image is segmented. As shown at 1706, the segments are now matched with respective fetal organ segments in the reference record and/or checked for compliance with one or more rules. For example, if the segments are as shown 1703, 1704, and 1705, namely depicting the intrahepatic tract of the umbilical vein on the anterior aspect, the stomach on the left side, and the spine with the transverse section of the abdominal aorta on the posterior aspect are identified in the image, for example as shown at FIG. 19, a match is found. Optionally, the match is based on the detection of a spot encircled by the abdominal circumference and the stomach. This allows, as shown at 1707, classifying the image as an image depicting the abdominal plane.

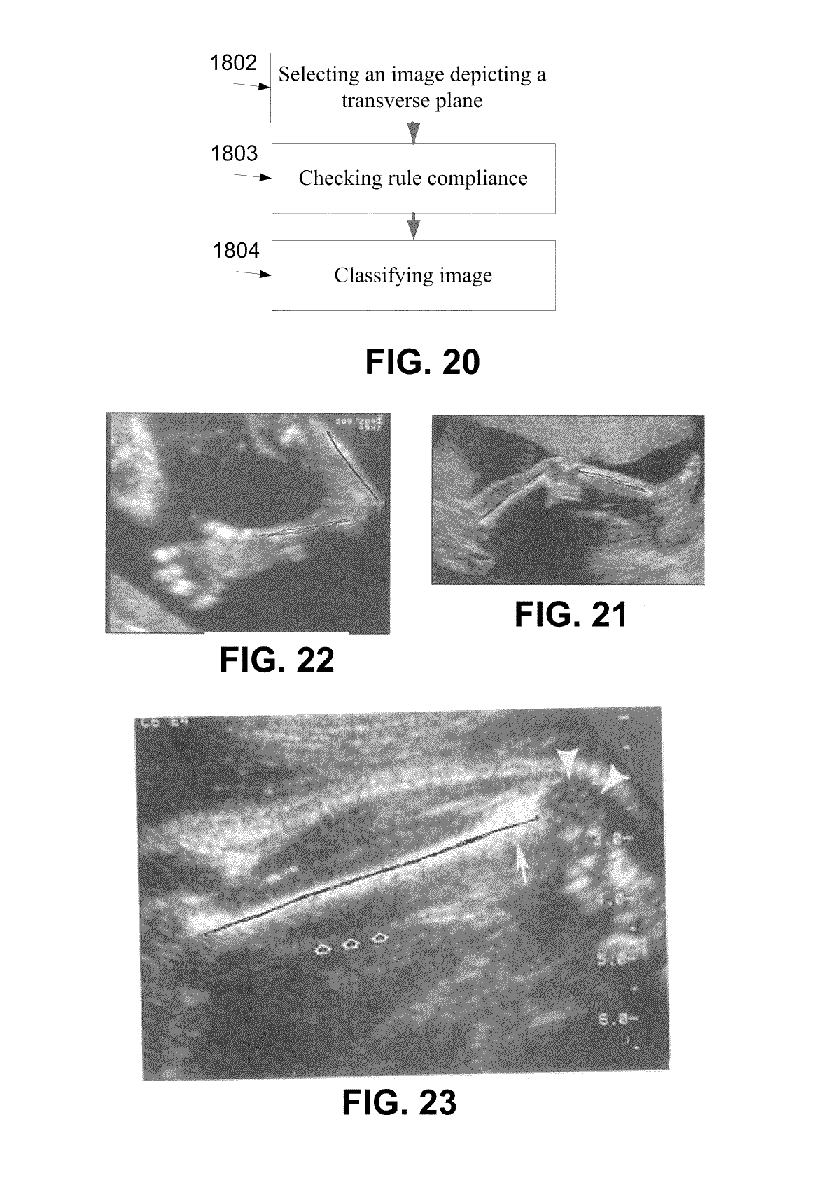

Reference is now made to FIG. 20, which is a flowchart of a process of identifying and classifying an image depicting a plane passing through a limb, for example the planes depicted in FIGS. 21-23, according to some embodiments of the present invention. First, as shown at 1802, an image is received. Then, the presence the femur, the humerus, the tibia and the radius therearound is detected, for example according to a match with segments having one or more of the following characteristics: a respective femur length; a respective humerus length; a respective tibia length; and a respective normal radius length. Accordingly, as shown at 1804, an image depicting an abdominal plane is detected and classified, for example a transverse plane at the level where the abdominal circumference may be measured.

It is expected that during the life of a patent maturing from this application many relevant systems and methods will be developed and the scope of the term ultrasonographic is intended to include all such new technologies a priori.

As used herein the term “about” refers to ±10%.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A method of monitoring ultrasonographic fetal organ screening, comprising:
receiving a plurality of ultrasonographic images captured by an ultrasonographic probe during an organ screening of a fetus;
automatically identifying a group of said plurality of ultrasonographic images, each member of said group depicting a fetal scan plane selected from a plurality of fetal scan planes;
presenting said group to an operator and receiving a diagnosis of each said member in response; and
monitoring said diagnosis to verify each said fetal scan plane being diagnosed.

2. The method of claim 1, wherein said identifying comprises identifying in each said member at least one fetal organ segment and classifying each said member as a certain of said plurality of fetal scan planes according to an analysis of said at least one fetal organ segment.

3. The method of claim 1, wherein said identifying comprises registering each said ultrasonographic image according to at least one reference map depicting at least one of said plurality of fetal scan planes.

4. The method of claim 1, wherein said identifying comprises evaluating at least one difference between said at least one fetal organ segment and at least respective fetal organ segment in at least one reference map depicting at least one of said plurality of fetal scan planes.

5. The method of claim 1, further comprising aligning said plurality of ultrasonographic images before said identifying.

6. The method of claim 1, wherein said receiving comprises receiving said plurality of ultrasonographic images while said ultrasonographic probe being manually maneuvered by a human operator over the uterus of a patient.

7. The method of claim 1, wherein said plurality of fetal scan planes are selected from of a group consisting of: a sagittal cerebral plane, a coronal cerebral plane, an axial transventricular plane, an axial transcerebellar plane, a four chamber cardiac view, a cardiac outflow tract, a great artery abdomen transverse cardiac plane, an transverse abdomen plane, a fetal spine plane, and a plane passing through the longitudinal axis of a fetal limb.

8. The method of claim 1, wherein said identifying comprises identifying said member as an image depicting a cerebral coronal plane by identifying a plurality of segments depicting the brain cavum septum pellucidum (CSP), the corpus callosum, and he anterior horns of the third ventricle of the brain of said fetus therein.

9. The method of claim 1, wherein said identifying comprises identifying said member as an image depicting a cerebral coronal plane by segmenting the brain cavum septum pellucidum (CSP) and the cavum of the brain of said fetus therein.

10. The method of claim 1, wherein said identifying comprises identifying said member as an image depicting a cerebral axial plane by segmenting the brain cavum septum pellucidum (CSP) and the Thalamus of the brain of said fetus therein.

11. The method of claim 1, wherein said identifying comprises identifying said member as an image depicting a cerebral axial plane by segmenting the Cerebellum and the Cisterna Magana of the brain of said fetus therein.

12. The method of claim 1, wherein said identifying comprises identifying said member as an image depicting a spinal axis plane by segmenting a plurality of vertebral bodies therein.

13. The method of claim 1, wherein said identifying comprises identifying said member as an image depicting a four-chambers plane by segmenting four heart chambers therein.

14. A method of managing ultrasonographic fetus organ screening, comprising:
receiving a plurality of ultrasonographic images captured by an ultrasonographic probe performing an organ screening of a fetus;
automatically identifying a group of said plurality of ultrasonographic images, each member of said group depicting a fetal scan plane selected from a plurality of fetal scan planes;
for each said member generating and presenting a form so as to allow an operator to submit a diagnosis of a respective said member.

15. A system of managing ultrasonographic fetus organ screening, comprising:
an interface which receives a plurality of ultrasonographic images captured by an ultrasonographic probe performing an organ screening of a fetus of a patient;
an plane selection module which automatically identifies a group of said plurality of ultrasonographic images, each member of said group depicting a fetal scan plane selected from a plurality of fetal scan planes; and
a form generator which generates a plurality of forms each for a respective said member, each said form being adapted to a fetal scan plane depicted by said respective member.

16. The system of claim 15, further comprising a presentation unit which generates an alert if said group does not include ultrasonographic images depicting all said plurality of fetal scan planes.

17. The system of claim 15, further comprising a user interface for allowing an operator to fill in said plurality of forms.

18. The system of claim 17, wherein said form generator monitors said filling in and outputs an indication about the completeness of said filling in.

19. The system of claim 15, further comprising a database having a plurality of reference maps of a plurality of normal fetal scan planes, said plane selection module compares between said plurality of reference maps and said plurality of ultrasonographic images to identify said group.

20. The system of claim 19, wherein each said reference map is associated with a different group of at least one of patients, organ screenings, and fetuses, said plane selection module compares said plurality of ultrasonographic images according to at least one of said organ screening, said fetus and said patient.

21. The system of claim 19, wherein each said reference map is associated with a different week of pregnancy.
